(19) United States
(12) Patent Application Publication
(10) Pub. No.: US 2017/0106035 A1
(43) Pub. Date: Apr. 20, 2017

(54) COMPOSITION FOR PREVENTING AND TREATING NON-ALCOHOLIC FATTY LIVER DISEASES

(71) Applicants: Hyoung-jin PARK, Gangwon-do (KR); MUSHMED CO., LTD., Gangwon-do (KR); JBK NATURAL MEDICINE INSTITUTE, Gyeonggi-do (KR)

(72) Inventors: Hyoung-jin PARK, Gangwon-do (KR); Bong-gun JANG, Gyeonggi-do (KR)

(21) Appl. No.: 15/317,752

(22) PCT Filed: Jun. 5, 2015
(86) PCT No.: PCT/KR2015/005664

§ 371 Date: Dec. 9, 2016

(30) Foreign Application Priority Data
Jun. 10, 2014 (KR) 10-2014-0070363

(51) Int. Cl. A61K 36/73; A61K 36/07; A23L 33/105; A61K 36/28

(52) U.S. Cl. CPC A61K 36/73; A61K 36/28; A61K 36/07; A23L 33/105; A23V 2002/00

(57) ABSTRACT

A composition includes a black chokeberry extract and at least one of a thistle extract, a black hoof mushroom extract and a mixture thereof. The composition is effective in preventing and treating non-alcoholic fatty liver disease caused by accumulation of fat in the liver due to excessive intake of nutrients or metabolic syndrome regardless of alcohol intake. The composition can improve the increase in liver weight, accumulation of neutral fat in the liver, increase in serum ALT concentration, and reduction of liver SOD activity and total antioxidant activity of the liver, in experimental animals grown with a high-fat diet. Since the fatty liver disease caused by a high-fat diet in experimental animals shows similar pathological and immunological manifestations to human non-alcoholic fatty liver disease, the composition can be provided as a pharmaceutical agent or health supplement food used to prevent and treat non-alcoholic fatty liver disease.

### TECHNICAL FIELD

The present disclosure relates to a composition which is effective in preventing and treating non-alcoholic fatty liver disease caused by accumulation of fat in the liver due to excessive intake of nutrients or metabolic syndrome regardless of alcohol intake.

The present application claims priority based on Korean Patent Application No. 10-2014-0070363 filed on Jun. 10, 2014, the entire contents of which are incorporated herein by reference.

### BACKGROUND ART

Fatty liver disease is a disease wherein fats are accumulated in the liver and is classified into alcoholic fatty liver disease caused by excessive alcohol intake and non-alcoholic fatty liver disease which is irrelevant to alcohol intake. The non-alcoholic fatty liver disease includes non-alcoholic hepatic steatosis and non-alcoholic steatotic hepatitis.

The non-alcoholic hepatic steatosis is pathologically classified into macrovesicular hepatic steatosis and microvesicular hepatic steatosis depending on the size of lipid droplets appearing in liver cells. Although the non-alcoholic hepatic steatosis is mostly macrovesicular, many microvesicles appear in the liver cells at earlier stage and macrovesicles are formed as they become larger as the disease develops.

Non-alcoholic fatty liver disease is closely related with excessive intake of nutrients and metabolic syndrome. The risk of non-alcoholic fatty liver disease is 10-15% for people with normal weight but increases greatly to 80% for overweight people. The prevalence rate of obesity in Korean adults has consistently increased from 26.3% in 1998 to 31.5% in 2005 in those aged 20 or older, and from 29.1% to 34.8% in those aged 30 or older over the same period. The prevalence rate of non-alcoholic fatty liver disease in diabetic patients reaches 50% and it is very highly correlated with insulin-resistant diabetes. Therefore, non-alcoholic fatty liver disease is emerging as an important disease in the modern society where metabolic syndromes such as obesity, etc. are increasing.

Because non-alcoholic hepatic steatosis proceeds chronically and slowly, there are no particular symptoms in early stages but the liver function is impaired as it gradually develops into non-alcoholic steatotic hepatitis. In extensive NASH, necrosis of liver cells occurs and fibrosis occurs as astrocytes are activated, notably at the terminal region of the hepatic vein. The fibrosis occurs in the liver in 15-50% of NASH patients and liver cirrhosis occurs in 30% of patients with liver fibrosis in 10 years. Therefore, non-alcoholic hepatic steatosis can lead to liver fibrosis or cirrhosis through NASH, if left untreated, and then to liver cancer.

Although it is necessary to inhibit the accumulation of fats in the liver in order to prevent non-alcoholic fatty liver disease, the only known method at present is calorie intake restriction and increase of calorie consumption through exercise.

### DISCLOSURE

### Technical Problem

The inventors of the present disclosure have conducted researches to develop a composition for preventing and treating non-alcoholic fatty liver disease. As a result, they have found out that a composition containing a black chokeberry (Aronia melanocarpa) extract and containing a thistle (Silybum marianum) extract or a black hoof mushroom (Phellinus linteus) extract exhibits a superior effect of preventing and treating non-alcoholic fatty liver disease and have completed the present disclosure.

Accordingly, the present disclosure is directed to providing a composition effective in preventing and treating non-alcoholic fatty liver disease, which contains a black chokeberry extract and a thistle extract or a black hoof mushroom extract.

### Technical Solution

The present disclosure provides a composition for preventing and treating non-alcoholic fatty liver disease, which contains a black chokeberry (Aronia melanocarpa) extract and comprises a thistle (Silybum marianum) extract, a black hoof mushroom (Phellinus linteus) extract or a mixture thereof.

The present disclosure also provides a pharmaceutical agent for preventing and treating non-alcoholic fatty liver disease, which contains the composition and a pharmaceutically acceptable carrier.

The present disclosure also provides a health supplement or health functional food for preventing and treating non-alcoholic fatty liver disease, which contains the composition and a sitologically acceptable carrier.

### Advantageous Effects

A composition of the present disclosure can greatly improve the increase in liver weight, accumulation of neutral fat in the liver, increase in serum ALT concentration, reduction of liver SOD activity, reduction of total antioxidant activity of the liver, etc. in experimental animals which are grown with a high-fat diet for a long period of time. Since the fatty liver disease caused by a high-fat diet in experimental animals shows similar pathological and immunological manifestations to human non-alcoholic fatty liver disease, the composition of the present disclosure can be provided as a superior pharmaceutical agent or health supplement food used to prevent and treat non-alcoholic fatty liver disease.

### BRIEF DESCRIPTION OF DRAWINGS

FIG. 1 shows the effect of a composition of the present disclosure on visual (top) and pathological (down) manifestations in the livers of experimental animals grown with a high-fat diet for 12 weeks. For investigation of the histological change of liver cells caused by fat accumulation, liver tissues were stained with Oil Red O or hematoxylin and eosin. NC: normal control group, HFC: high-fat diet control group, APS: group to which the composition of the present disclosure was administered.

FIG. 2 shows the effect of a composition of the present disclosure on the liver weight of experimental animals grown with a high-fat diet for 12 weeks. NC: normal control group, HFC: high-fat diet control group, A: group to which a composition containing a black chokeberry extract alone was administered, AP: group to which a composition containing a black chokeberry extract and a black hoof mushroom extract was administered, AS: group to which a composition containing a black chokeberry extract and a thistle extract was administered, APS: group to which a composition containing a black chokeberry extract, a black hoof mushroom extract and a thistle extract was administered. : significantly (p<0.001) higher as compared to NC, ‡: significantly (p<0.01) lower as compared to HFC, ★: significantly (p<0.01) lower as compared to HFC and A.

FIG. 3 shows the effect of a composition of the present disclosure on the neutral fat content in the livers of experimental animals grown with a high-fat diet for 12 weeks. NC: normal control group, HFC: high-fat diet control group, A: group to which a composition containing a black chokeberry extract alone was administered, AP: group to which a composition containing a black chokeberry extract and a black hoof mushroom extract was administered, AS: group to which a composition containing a black chokeberry extract and a thistle extract was administered, APS: group to which a composition containing a black chokeberry extract, a black hoof mushroom extract and a thistle extract was administered. : significantly (p<0.001) higher as compared to NC, ‡: significantly (p<0.05) lower as compared to HFC, ★: significantly (p<0.05) lower as compared to HFC and A.

FIG. 4 shows the effect of a composition of the present disclosure on the serum ALT concentration of experimental animals grown with a high-fat diet for 12 weeks. NC: normal control group, HFC: high-fat diet control group, A: group to which a composition containing a black chokeberry extract alone was administered, AP: group to which a composition containing a black chokeberry extract and a black hoof mushroom extract was administered, AS: group to which a composition containing a black chokeberry extract and a thistle extract was administered, APS: group to which a composition containing a black chokeberry extract, a black hoof mushroom extract and a thistle extract was administered. : significantly (p<0.001) higher as compared to NC, ‡: significantly (p<0.01) lower as compared to HFC, ★: significantly (p<0.01) lower as compared to HFC and A.

FIG. 5 shows the effect of a composition of the present disclosure on the liver SOD activity of experimental animals grown with a high-fat diet for 12 weeks. NC: normal control group, HFC: high-fat diet control group, A: group to which a composition containing a black chokeberry extract alone was administered, AP: group to which a composition containing a black chokeberry extract and a black hoof mushroom extract was administered, AS: group to which a composition containing a black chokeberry extract and a thistle extract was administered, APS: group to which a composition containing a black chokeberry extract, a black hoof mushroom extract and a thistle extract was administered. : significantly (p<0.001) higher as compared to NC, ‡: significantly (p<0.001) lower as compared to HFC, ★: significantly (p<0.001) lower as compared to HFC and A.

FIG. 6 shows the effect of a composition of the present disclosure on the total antioxidant activity of the livers of experimental animals grown with a high-fat diet for 12 weeks. NC: normal control group, HFC: high-fat diet control group, A: group to which a composition containing a black chokeberry extract alone was administered, AP: group to which a composition containing a black chokeberry extract and a black hoof mushroom extract was administered, AS: group to which a composition containing a black chokeberry extract and a thistle extract was administered, APS: group to which a composition containing a black chokeberry extract, a black hoof mushroom extract and a thistle extract was administered. : significantly (p<0.001) higher as compared to NC, ‡: significantly (p<0.05) lower as compared to HFC, ★: significantly (p<0.001) lower as compared to HFC and A.

### BEST MODE FOR CARRYING OUT INVENTION

The present disclosure provides a composition for preventing and treating non-alcoholic fatty liver disease, which contains a black chokeberry (Aronia melanocarpa) extract and contains a thistle (Silybum marianum) extract or a black hoof mushroom (Phellinus linteus) extract.

Black chokeberry (Aronia melanocarpa) is a perennial shrub belongs taxonomically to the family Rosaceae. It is known to contain anthocyanins and catechins at the highest contents among the berries found until now. Anthocyanins are natural pigment glycosides present mainly in the flowers, fruits, leaves and stems of plants. Anthocyanins are found at high concentrations mainly under intense UV light, harsh coldness and high humidity, which may be because the UV-absorbing anthocyanins are produced on the surface or in intermediate layers of plants.

Specifically, in the present disclosure, the black chokeberry extract may be a pulp extract of the shrub which contains anthocyanins at high contents. The extract may be a compression extract of pulp or a solvent extract using an extraction solvent. The extraction solvent may be water or a C₁-C₄ alcohol. Specifically, ethanol may be used.

Thistle (Silybum marianum) is a perennial plant in the family Asteraceae which grows wildly in the mountains and fields throughout Korea. It is used as in medicine and is known to be effective in hemostasis, detoxification and anti-inflammation. In particular, there exist various flavonolignan compounds in the thistle extract, including silybin, isosilybin, silychristin, silydianin, etc.

Specifically, in the present disclosure, the thistle extract may be a seed or pulp extract of thistle. More specifically, a seed extract extracted from dried seeds may be used. The extraction solvent may be water or a C₁-C₄ alcohol. Specifically, ethanol may be used.

Black hoof mushroom (Phellinus linteus), also called woody mud mushroom, is a perennial wood-decaying mushroom growing on mulberry tree, etc. It resembles a clot of mud in early years and a projecting tongue after it is fully grown. So, it is also called tree tongue. β-Glucan, which is a polysaccharide extracted from black hoof mushroom, is known to have immunoregulatory activity, etc.

Specifically, in the present disclosure, the black hoof mushroom extract may be a fruit body extract extracted from the dried fruit body. The extraction solvent may be water or a C₁-C₄ alcohol. Specifically, hot water may be used.

The composition of the present disclosure contains the black chokeberry extract and contains the thistle extract, the black hoof mushroom extract or a mixture thereof. Specifically, it may contain all of the black chokeberry extract, the thistle extract and the black hoof mushroom extract.

The composition of the present disclosure may contain specifically 10-500 parts by weight, more specifically 30-300 parts by weight, most specifically 50-200 parts by weight, of each of the thistle extract or the black hoof mushroom extract based on 100 parts by weight of the black chokeberry extract.

The composition of the present disclosure suppresses the increase in liver weight caused by a high-fat diet and suppresses the increase in neutral fat content in the liver caused by a high-fat diet. In addition, the composition of the present disclosure suppresses the increase in serum alanine transaminase (ALT) concentration caused by a high-fat diet, improves the liver superoxide dismutase (SOD) activity suppressed by a high-fat diet and improves the total antioxidant activity of the liver suppressed by a high-fat diet.

Accordingly, the composition of the present disclosure may be used to prevent and treat non-alcoholic fatty liver disease which is accompanied by excessive intake of nutrients, metabolic syndrome, etc.

Also, the composition of the present disclosure may be prepared into a pharmaceutical agent together with a pharmaceutically acceptable carrier. A daily dosage of the pharmaceutical agent may be controlled adequately within a range of 5-100 mg/kg depending on the contents of anthocyanin, silymarin and β-glucan which are marker ingredients of the respective extracts. The pharmaceutical agent may be administered orally and may be formulated as a tablet, a capsule or a liquid. The pharmaceutical agent may contain various excipients, diluents, etc. known to those of ordinary skill in the art to which the present disclosure belongs.

The composition of the present disclosure may also be prepared into a health supplement or health functional food for preventing and treating non-alcoholic fatty liver disease together with a sitologically acceptable carrier.

Hereinafter, the present disclosure will be described in detail through examples. However, they are provided for illustrative purposes only and should not be construed as limiting the scope of the present disclosure.

### PREPARATION EXAMPLE

### Preparation of Extracts

A compression extract was obtained by compressing and extracting the pulp of frozen black chokeberry and removing precipitates. An ethanol extract was obtained by mixing the remaining residue with 50 wt % ethanol at a weight ratio of 1:2 and extracting for 2 hours at room temperature. A black chokeberry extract was prepared by clarifying a mixture of the compression extract and the ethanol extract using an Alfa Laval disc-type separator, concentrating through distillation and then preparing into powder using a spray dryer.

The dried seed of thistle was added to ethanol of 2 times based on weight and solid contents were removed by extracting at room temperature for 6 hours. Then, a thistle extract was obtained by preparing into powder using a spray dryer.

The dried fruit body of black hoof mushroom was added to distilled water of 2 times based on weight. After heating at 100° C. for 12 hours, solid contents were removed through filtration. Then, a black hoof mushroom extract was prepared through concentration at 50° C. under reduced pressure followed by freeze-drying.

### TEST EXAMPLE 1

### Induction of Hepatic Steatosis in Experimental Animals and Administration of Composition

When experimental animals are grown with a high-fat diet for a long period of time, fatty liver disease occurs as fats are accumulated in the liver. The high-fat diet-induced fatty liver disease shows very similar pathological and immunological manifestations to human non-alcoholic fatty liver disease (Journal of Hepatology (2011), doi:10.1016/j.jhep, 2010.08.19). Therefore, this model was used to demonstrate the effectiveness of the composition in non-alcoholic fatty liver disease.

5-week-old male C57BL/6 mice were used for experiments after accustoming to an environment of room temperature and constant humidity for 1 week.

For a normal control group (NC), the experimental animals were grown with a normal feed (containing 12 kcal % crude fat) while orally administering 0.1 mL of distilled water once a day through the experiment.

For high-fat diet groups, the experimental animals were grown with a high-fat diet feed (containing 60 kcal % crude fat) for 12 weeks to induce hepatic steatosis. They were divided into a control group (HFC) and test groups.

0.1 mL of distilled water for the control group and each composition dissolved in 0.1 mL of distilled water for the test groups were orally administered once a day through the experiment.

The test groups were subdivided into a group to which a composition containing the black chokeberry extract alone was administered (A); a group to which a composition containing a mixture of the black chokeberry extract and the black hoof mushroom extract was administered (AP); a group to which a composition containing a mixture of the black chokeberry extract and the thistle extract was administered (AS); and a group to which a composition containing a mixture of the black chokeberry extract, the black hoof mushroom extract and the thistle extract was administered (APS). To the experimental animals of the A administration group, 50 mg/kg body weight of the black chokeberry extract was administered every day. To the experimental animals of the AP administration group, 50 mg/kg body weight of the black chokeberry extract and 50 mg/kg body weight of the black hoof mushroom extract were administered every day. To the experimental animals of the AS administration group, 50 mg/kg body weight of the black chokeberry extract and 50 mg/kg body weight of the thistle extract were administered every day. And, to the experimental animals of the APS administration group, 50 mg/kg body weight of the black chokeberry extract, 50 mg/kg body weight of the black hoof mushroom extract and 50 mg/kg body weight of the thistle extract were administered every day.

### TEST EXAMPLE 2

### Visual and Pathological Examination of Liver

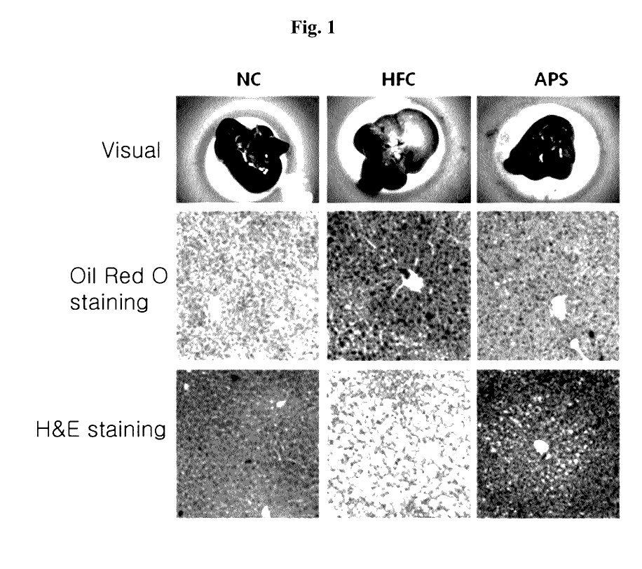

At 12 weeks after the experiment was started, livers were extracted from the experimental animals and changes were observed visually and histologically. The result is shown in FIG. 1.

The liver of the high-fat diet control group (HFC) was found to be enlarged and yellowed as compared to that of the normal control group (NC). In contrast, the liver of the group to which the composition containing a mixture of the black chokeberry extract, the black hoof mushroom extract and the thistle extract was administered (APS) showed no enlargement or color change.

When the fats accumulated in liver cells were stained with Oil Red O, the liver cells of the NC group showed no lipid droplets whereas the liver cells of the HFC group showed large lipid droplets stained red around the small vein. The liver cells of the APS administration group showed only small lipid droplets stained red. This pattern was also observed in the liver cells stained with hematoxylin and eosin. That is to say, the liver cells of the HFC group showed macrovesicles formed as fats are removed whereas the liver cells of the APS administration group showed microvesicles rather than macrovesicles.

This result suggests that the mixed composition (APS) significantly suppresses liver enlargement and appearance of large lipid droplets in liver cells caused by the high-fat diet.

### TEST EXAMPLE 3

### Effect on Liver Weight

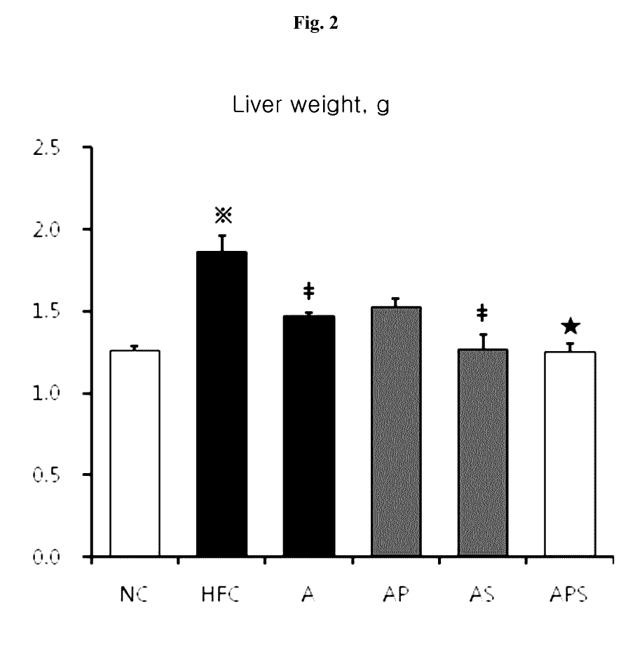

The liver weight of the experimental animals 12 weeks after the experiment was started is shown in FIG. 2. The liver weight of the high-fat diet control group (HFC) was increased by 47.81% as compared to that of the normal control group (NC). The liver weight of the A administration group, the AP administration group, the AS administration group and the APS administration group was decreased by 21.12%, 18.12%, 31.92% and 32.81%, respectively, as compared to that of the HFC group.

It was confirmed from this experiment that the mixed composition (APS) suppresses the increase in liver weight caused by the high-fat diet the most strongly.

### TEST EXAMPLE 4

### Effect on Neutral Fat Content in Liver

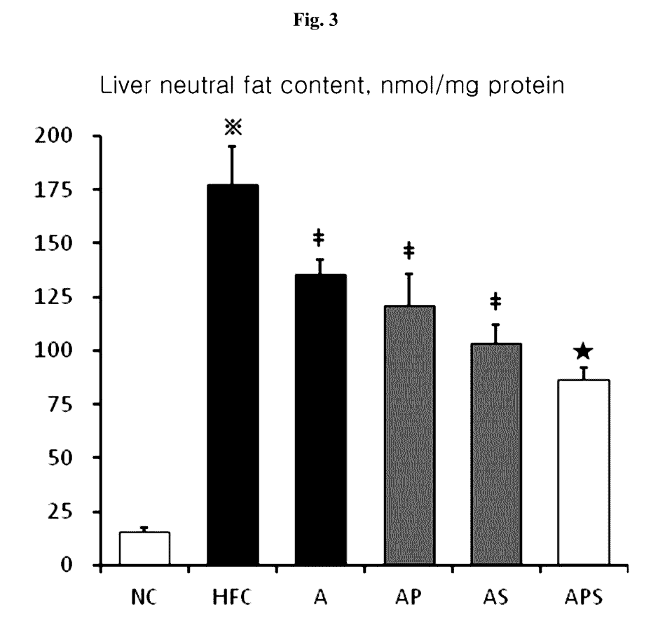

The neutral fat content in the liver tissues of the experimental animals 12 weeks after the experiment was started is shown in FIG. 3. The neutral fat content in the liver of the high-fat diet control group (HFC) was increased by 1,076.12% as compared to that of the normal control group (NC). The neutral fat content in the liver of the A administration group, the AP administration group, the AS administration group and the APS administration group was decreased by 23.44%, 31.72%, 41.78% and 51.43%, respectively, as compared to that of the HFC group.

It was confirmed from this experiment that the mixed composition (APS) suppresses the increase in neutral fat content in the liver caused by the high-fat diet the most strongly.

### TEST EXAMPLE 5

### Effect on Serum ALT Concentration

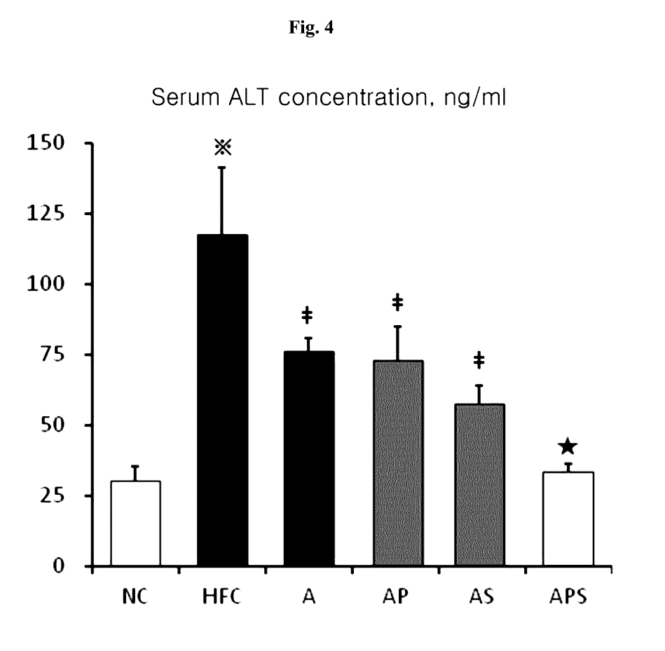

The ALT concentration measured from the serum of the experimental animals 12 weeks after the experiment was started is shown in FIG. 4. The serum ALT concentration of the high-fat diet control group (HFC) was increased by normal 290.73% as compared to that of the normal control group (NC). The serum ALT concentration of the A administration group, the AP administration group, the AS administration group and the APS administration group was decreased by 35.33%, 38.04%, 51.06% and 71.50%, respectively, as compared to that of the HFC group.

It was confirmed from this experiment that the mixed composition (APS) suppresses the increase in serum ALT concentration caused by the high-fat diet the most strongly.

### TEST EXAMPLE 6

### Effect on Liver SOD Activity

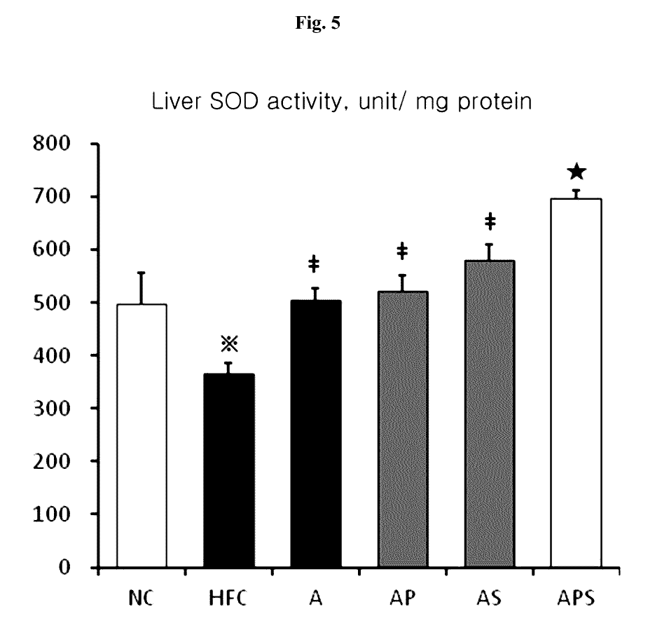

The SOD activity in the liver tissues of the experimental animals 12 weeks after the experiment was started is shown in FIG. 5. The liver SOD activity of the high-fat diet control group (HFC) was decreased by 26.54% compared to that of the normal control group (NC). The liver SOD activity of the A administration group, the AP administration group, the AS administration group and the APS administration group was increased by 37.79%, 42.97%, 58.72% and 90.78%, respectively, as compared to that of the HFC group.

It was confirmed from this experiment that the mixed composition (APS) increases the liver SOD activity decreased by the high-fat diet the most strongly.

### TEST EXAMPLE 7

### Effect on Total Antioxidant Activity of Liver

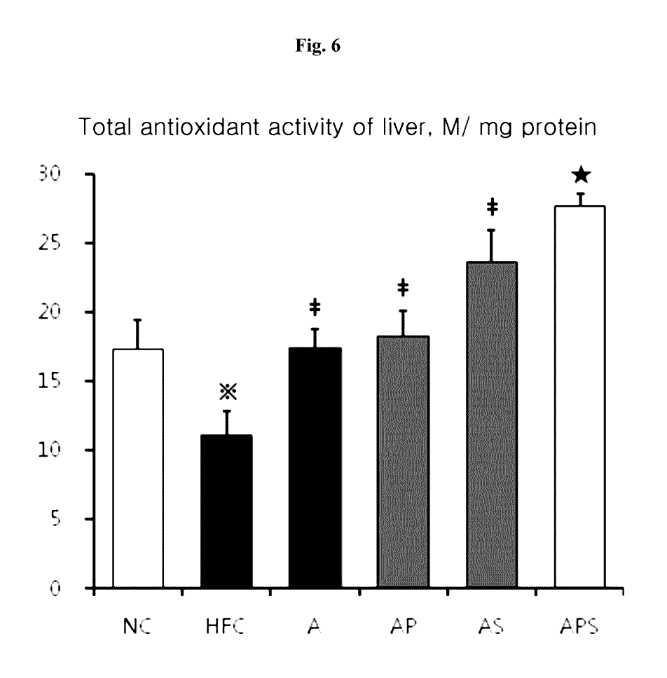

The total antioxidant activity of the liver tissues of the experimental animals 12 weeks after the experiment was started is shown in FIG. 6. The total antioxidant activity of the liver was decreased by 36.12% compared to that of the normal control group (NC). The total antioxidant activity of the liver of the A administration group, the AP administration group, the AS administration group and the APS administration group was increased by 39.56%, 74.47%, 113.62% and 150.26%, respectively, as compared to that of the HFC group.

It was confirmed from this experiment that the mixed composition (APS) increases the total antioxidant activity decreased by the high-fat diet the most strongly.

It was confirmed from these experiments that the composition of the present disclosure decreases the increased liver weight and liver neutral fat content in experimental animals grown with a high-fat diet and suppresses the increase in serum ALT concentration. This suggests that the composition suppresses accumulation of fats in the liver caused by the high-fat diet and prevents damage of liver function.

In addition, because it was confirmed that the composition of the present disclosure greatly increases the liver SOD activity and total antioxidant activity of the liver suppressed by a high-fat diet, the composition of the present disclosure is thought to greatly improve the antioxidant activity of the liver and prevents development into steatotic hepatitis by decreasing fat accumulation in the liver caused by the high-fat diet.

Those skilled in the art will appreciate that the conceptions and specific embodiments disclosed in the foregoing description may be readily utilized as a basis for modifying or designing other embodiments for carrying out the same purposes of the present disclosure. Those skilled in the art will also appreciate that such equivalent embodiments do not depart from the spirit and scope of the disclosure as set forth in the appended claims.

1. A composition for preventing and treating non-alcoholic fatty liver disease, comprising a black chokeberry (Aronia melanocarpa) extract and at least one of a thistle (Silybum marianum) extract, a black hoof mushroom (Phellinus linteus) extract and a mixture thereof.

2. The composition for preventing and treating non-alcoholic fatty liver disease according to claim 1, wherein the thistle extract is an extract extracted from the seed of thistle and the black hoof mushroom extract is an extract extracted from the fruit body of black hoof mushroom.

3. The composition for preventing and treating non-alcoholic fatty liver disease according to claim 1, which comprises 10-500 parts by weight of the thistle extract based on 100 parts by weight of the black chokeberry extract.

4. The composition for preventing and treating non-alcoholic fatty liver disease according to claim 1, which comprises 10-500 parts by weight of the black hoof mushroom extract based on 100 parts by weight of the black chokeberry extract.

5. A pharmaceutical agent for preventing and treating non-alcoholic fatty liver disease, which comprises the composition according to claim 1 and a pharmaceutically acceptable carrier.

6. A healthupplement or health functional food for preventing and treating non-alcoholic fatty liver disease, which comprises the composition according to claim 1 and a sitologically acceptable carrier.
